(19) United States
(12) Patent Application Publication
(10) Pub. No.: US 2021/0313032 A1
(43) Pub. Date: Oct. 7, 2021

(54) SYSTEMS AND METHODS FOR DYNAMIC INTERACTIVE DRUG SAVINGS REPORTS SERVICES

(71) Applicant: Gemini Health LLC, Sausalito, CA (US)

(72) Inventors: Edward FOTSCH, Sausalito, CA (US); Roger PINSONNEAULT, Johns Creek, GA (US); Dwight DAVIS, Little Rock, AR (US)

(21) Appl. No.: 17/224,894

(22) Filed: Apr. 7, 2021

Related U.S. Application Data
(60) Provisional application No. 63/006,229, filed on Apr. 7, 2020.
(60) Provisional application No. 63/067,514, filed on Aug. 19, 2020.

(51) Int. Cl. G16H 20/10; G06Q 30/06; G16H 10/60; G06F 16/2453; G06Q 30/02

(52) U.S. Cl. CPC G16H 20/10; G06Q 30/0629; G06Q 30/0283; G06F 16/24532; G16H 10/60

(57) ABSTRACT

Described herein are methods and systems for controlling the operation of one or more prescription cost savings systems, including methods and systems for the interactive and dynamic generation of Drug Savings Reports.

### CROSS REFERENCE TO RELATED APPLICATIONS

This patent application claims priority to U.S. Provisional Patent Application No. 63/006,229, filed on Apr. 7, 2020, and titled “SYSTEMS AND METHODS FOR DYNAMIC INTERACTIVE DRUG SAVINGS REPORTS SERVICES,” as well as U.S. Provisional Patent Application No. 63/067,514, filed on Aug. 19, 2020, and titled “SYSTEMS AND METHODS FOR DYNAMIC INTERACTIVE DRUG SAVINGS REPORTS SERVICES,” each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods and systems for prescription selection for streamlining prescription pricing, and more specifically to systems to identify and prioritize therapeutic prescription alternatives to be created interactively with managed care pharmacists and other end-users responsible for managing drug and related healthcare costs.

### BACKGROUND

Health costs continue to grow in the U.S. and are well documented as a formidable national challenge, with prescription drug costs among the leading sources of cost escalation. Currently, Prescribers are typically notified to renew existing prescriptions for their patients by phone call, fax or electronic systems when an existing prescription has expired, but cost information is generally not provided to the Prescriber, even if the cost of the medication has gone up dramatically and/or lower cost therapeutically equivalent medications are available. Alternative drug or fulfillment cost information is not routinely or automatically delivered to Prescribers or patients related to prescribing new medications or for evaluating the cost-effectiveness of existing medications. Prescribers cannot consider cost-efficiency when prescribing or renewing prescription, or at the point of care. Drug costs and cost savings opportunities are not currently considered for new or existing prescriptions, and as a result drug costs continue to escalate.

For most drug categories there are numerous drug alternatives that a Prescriber might consider in treating a patient. Many Prescribers are not completely familiar with all drugs in every category in which they prescribe. And since Prescribers are given neither a list of alternatives to consider, nor the cost of alternative drugs, both clinical and economic efficiency are lacking.

Drug costs can vary by many factors including: drug chosen, form of drug (long acting, etc.), patient's insurance, patient's deductible, including high deductible plans and Affordable Care Act (ACA) health plans, pharmacy and patient financial assistance programs. Drug costs are dynamic and change regularly, even between new scripts and renewal scripts. Drug costs may also vary depending on the fulfillment means used: e.g., $4 generics (Wal-Mart, Target, etc.), pharmacy discount card or mail order, etc. The pricing of a medication can be complicated.

The optimal alternative drug for a given patient may vary substantially based upon Payer (e.g., insurer, health organizations, etc.) formularies, Payer preferences and provider preferences, or because new drugs are approved or new uses of existing medications are found or approved.

A prescription drug and the pharmacy used to fill the prescription may be selected via an e-Prescribing or electronic health record (“EHR”) application. For example, a Prescriber may select the drug to be prescribed. The preferred pharmacy (e.g., a ‘pharmacy of last fill’) may be a default field in electronic prescribing workflow. The patient's preferred pharmacy is typically entered into the provider's EHR or ePrescribing application when the patient initially registers with the provider for medical care. The prescription may be sent electronically to the patient's chosen pharmacy.

While Prescribers have shown an interest in knowing drug costs, alternative drugs and their costs, and related clinical messages about the prescriptions to be created or renewed, Prescribers typically do not want to spend time considering all possible therapeutic alternatives and then price-shopping for each drug for each patient. Prescribers often have more than one drug in a specific clinical category that they use to treat a specific illness; such as using any of a number of different Statins to treat high cholesterol. Prescribers in these instances could select drugs that are more cost effective for the patient when a prescription is created or renewed if alternative medications and costs were provided.

Although various methods and systems for reducing drug costs have been proposed, the various components that contribute the actual cost of drugs are complex and distributed. For example, actual drug cost may include not just the cost paid by the patient, but the cost of patient's insurance (including copays and deductibles), the cost to the provider (e.g., the physician, the cost to the pharmacy, as well as the costs to the insurer. Although attempts to aggregate these various components and costs have been attempted, this information is also distribute between various systems and databases for which access may be limited and expensive.

There is a pressing need for identification and selection of therapeutic drug alternatives for existing medications, ideally at the time and point of care. In particular it would be beneficial to provide methods and apparatuses (e.g., systems) that may streamline accessing, collecting and processing so that drug alternatives, including pricing information, can be provided to patients, providers and/or insurers at a reasonable cost and within a reasonable amount of time.

### SUMMARY OF THE DISCLOSURE

Described herein are methods and systems for controlling the operation of one or more prescription cost savings systems, including methods and systems for the interactive and dynamic generation of Drug Savings Reports. Specifically, described herein are methods and systems that enable the efficient and cost-effective creation of Drug Savings Reports (“DSRs”) (by Pharmacist or other authorized users) to identify therapeutic alternatives for existing prescription medications including costs, from a variety of distributed third party sources of information, including incorporating Pharmacist, Payer coverage rules and provider preferences. These systems and methods (e.g., computer-operated methods, including software) may aggregated, configure and schedule inquiries (“queries”) into a diverse set of third-party databases in order to generate on or a number (including very large numbers) of DSRs that may include current medications as found in payer claims files and may include them in the DSR for medication reconciliation purposes including comparison to the EHR-based medication list. These systems may also be configured to incorporate pharmacist and provider preferences and in particular, may allow interactive inclusion of alternative drugs that are always presented or always excluded either in general or on a patient-specific basis. Preferences may be identified automatically through claims data or cost transparency transactions to include alternatives selected by Providers and those not selected. The DSR may also identify a lower cost pharmacy for the patient to fill existing medications including the ability for pharmacies to interactively insert coupons or rewards into the DSR to motivate patients to change to the new pharmacy. The DSR may include a Shared Patient Savings (SPS) payment or reward for the patient that further aligns the financial interest of the patient with that of the Plan to optimize the use of cost-effective medications.

The methods and systems described herein may be useful with and may improve upon, those described in U.S. patent application Ser. No. 16/036,851, filed on Jul. 16, 2018, and titled “METHODS AND APPARATUSES FOR PROVIDING ALTERNATIVES FOR PREEXISTING PRESCRIBED MEDICATIONS”, herein incorporated by reference in its entirety.

In some examples, these systems may actively monitor a plurality of third-party data bases (e.g., databases that are owned, run and/or administered by a variety of institutional and private third parties) in a rules-based manner with interactive rules configurable by Pharmacist, Prescribers or Payers to identify over time new opportunities for switches from existing medications to lower cost, clinically equivalent medications and automatically generate new DSRs when these opportunities arise with subsequent automated notification of Pharmacist. Databases may include payer claims, formularies, provider directories and patient lists attributed to specific providers.

The list of existing medications comes from the Payer claims file (i.e., medications that have been filled by the patient), but a list can also come from an Electronic Health Record (“EHR”). If available from both sources the DSR can identify which listed drugs come from which source and allow the Pharmacist and Prescribers to compare the lists through a process sometimes known as medication reconciliation.

The systems and methods for dynamic interactive DSR services, including dynamic generation of Drug Savings Reports, can identify alternative drugs from a Configurable Alternatives engine that identifies therapeutic equivalent alternative drugs then presents the alternatives to the Pharmacist or provider based upon Payer rules and Pharmacist or provider preferences, including alternatives that should always be presented in response to an existing medication or never presented either in general or for a specific patient. Preferences may be identified automatically through claims data or cost transparency transactions, include alternatives selected by Providers, and those not selected.

Assembling DSR data involves the transfer of an enormous amount of data across multiple digital platforms. Such high-volume data transfer taxes system resources for both the data holders and the DSR platform. In order to address this challenge, the system allows for the batching and scheduling of the data transfers to times of lower active system utilization ensuring data transfers are within system resource utilization limitations and are compliant with data holder system usage agreements.

Assembling DSR data also involves the challenge of matching data from multiple platforms and sources into a unified patient data file. In order to address this challenge, the system utilizes a dynamic matching logic that is able to identify, link, and organize these multiple data sources into a unified data file which is incorporated into the DSR.

DRS data also presents a challenge to users in the sheer quantity of data being communicated. In order to transmit the data in the most advantageous format to end users, the system utilizes scoring logic to prioritize the data. The system is able to score and prioritize data based on the highest opportunity for savings utilizing a series of system developed metrics including drug-specific savings per transaction combined with the historic drug-specific frequency of the transaction. The resulting data presentation allows for a better utilization of resources toward the greatest opportunity for success.

The DSRs provide additional innovation in that they can be interactively configured, requested, viewed, downloaded, sorted, triaged, edited, forwarded to Patients or Prescribers, placed in a pending file and returned for confirmation of switches by the Pharmacist. These interactive features exist within multiple layers of the system, ranging from the original data queries to the preferences of the end user.

Customizable DSR sorting and filtering can include variables including; prescriber, drug, type of savings, date of last fill, amount of patient savings and amount of total savings.

The interactive DSRs can be customized and edited by the pharmacists by inserting or selecting from a list of preferred alternative drugs which would then change the medication alternatives presented, including recalculating SPS amounts if applicable.

Pharmacists can set custom reminders to revisit one or more DSRs. For example, a pharmacist might set a reminder to review a DSR shortly before the end of a 90-day prescription fill period or upon the return of a prescriber from PTO.

The systems and methods for dynamic interactive DSR services including dynamic generation of Drug Savings Reports can facilitate transmission of DSRs from the pharmacists to the prescriber as well as related communication and scheduling of communication.

The DSRs can be communicated by the pharmacists to the prescriber via fax, email, via the interactive DSR portal including integration into video conferencing, or directly into the prescriber's EHR. In each case, the DSR can be reviewed and communicated back to the pharmacist with switches to lower cost alternatives authorized by the prescriber; allowing the pharmacists to change the prescription, the pharmacy, and notify the patient of the change. The video delivery and discussion of the DSR with the prescriber can also include a recording of the discussion to create a medical-legal record.

The systems and methods for dynamic interactive DSR services, including dynamic generation of Drug Savings Reports, can include scheduling components that syncs to the prescriber's calendar, the EHR, and to the pharmacist's calendar inside or outside of the DSR portal.

The systems and methods for dynamic interactive DSR services, including dynamic generation of Drug Savings Reports, can not only reduce the cost of prescriptions for patients, payers and provider groups, but also benefit a Patient by increasing adherence to medication and preventing delayed onset of care, thereby lowering overall healthcare costs. The systems and methods for dynamic interactive DSR services can further enable a prescriber to focus primarily on patient care, while their Pharmacists colleague uses pharmacy expertise to identify, triage, recommend appropriate lower cost alternative medications, and implement changes to lower cost alternatives once authorized by the prescriber.

The system and method for dynamic interactive DSR services can calculate the annual savings to the Patient and to the Payer and/or provider group at risk that would be achieved by substituting a lower cost clinically equivalent alternative medication for an existing medication and presents this information to the Pharmacists. The system and method also take the total savings potential represented by all alternative drugs and provides the Pharmacist with an estimate of the total potential savings achievable by using all available lower cost alternatives.

The system and method can also create additional interactive financial incentive that benefits the Patient associated with substituting a lower cost medication by taking a predetermined portion of the Payer savings and delivering it to the Patient in the form of a credit or copay reduction (“Shared Patient Savings” or “SPS”). SPS can be dynamic based upon the ratio of patient savings to payer savings for a given alternative. SPS can also be customized based upon Pharma manufacturer's patient financial assistance program that can reduce or eliminate patient costs and, therefore, patient savings when moving to a lower cost alternative. The redemption of the SPS can be affected either electronically via the system and method's communication with the Payer's claims processor and/or via a printable SPS coupon provided to the Patient.

For example, the system and method for dynamic interactive DSR services can use an internal proprietary configurable database and rules engine to identify potential lower cost alternative drugs that the prescriber might consider in treating the Patient. The system and method can then rank and prioritize these alternatives based upon interactive criteria such as Payer formularies, Payer preferences, Pharmacists preferences, Prescriber preferences. It includes an interactive means of communications related to DSRs, patient clinical status and also medications previously taken (“MPT”) by the patient. This interactive prioritization of alternatives can also include alternatives that should always be presented in response to an existing medication, or never be presented either in general or for a specific patient.

The system and method can determine the Patient's pharmacy benefits manager (“PBM”) and sends customizable data regarding the Patient and the prescriber to the Patient's PBM requesting specific information regarding options for lower cost medications. The system and method can monitor changes in the patient's medications, lower cost therapeutic drug alternatives available such as new drugs, drugs going off patent, changes in formularies, changes in rebates, changes in pharmacy coverage and members, or changes in providers who are members of a medical group. It allows member or provider-defined time intervals to automatically generate Drug Savings Reports with predetermined recipients based upon these changes. The system and method can further deliver to the Pharmacists via an interactive portal the exact cost of each relevant alternative drug inclusive of total drug costs, as well as the Patient's out-of-pocket costs inclusive of Patient's insurance, deductibles, copays and other relevant factors.

The system and method for dynamic interactive DSR services can allow the Pharmacists to interactively review lower cost alternatives presented in the DSR, customize the alternatives, and then request that the DSR be re-generated with the new Pharmacist-selected alternatives and their corresponding costs and savings.

The systems and methods for dynamic interactive DSR services, including dynamic generation of Drug Savings Reports, can identify alternative drugs from a Configurable Alternatives engine that identifies therapeutic equivalent alternative drugs, and then presents the alternatives to the Pharmacist or provider based upon Payer rules and Pharmacist or provider preferences and customizations. Preferences and customizations may be identified by the prescriber themselves, a pharmacist or other intermediary, or automatically through claims data or cost transparency transactions.

The system and method for dynamic interactive DSR services can allow the Pharmacists to review custom lower cost alternatives using the DSR, including interactivity noting or excluding medications previously taken (“MPT”) by the patient based upon Pharmacists or payer selected preferences.

The system and method for dynamic interactive DSR services allows the Pharmacists to select an alternative diagnosis and then request lower cost alternatives presented in the DSR based upon the selected alternative diagnosis.

The system and method for dynamic interactive DSR services allows the Pharmacists to request DSR files based upon specific Prescribers, Patients, Drugs or Savings potentials. The Pharmacist can then download an interactive file of DSRs and work offline. Alternatively, the Pharmacist could review, triage, modify and rerun or mark as pending DSRs via the portal without a file download. The Pharmacists can also interactively annotate the DSRs, noting which alternatives they have recommended to Prescribers for switches from current medications. The Pharmacist may then upload the DSRs via the portal for subsequent confirmation of switches that were made as confirmed by the Payer's claims file.

The system and method for dynamic interactive DSR services allows the Pharmacists to communicate DSRs to specific Prescribers via fax, email, via the DSR portal, in EHRs, or via other interactive means, and with the ability for the prescriber to authorize specific switches to lower cost alternatives and resubmit the DSR to the pharmacist. The Pharmacist is then authorized to contact the pharmacy to change the medication to the lower cost alternative and also contact the patient confirming the switch.

The systems and methods for dynamic interactive DSR services, including dynamic generation of Drug Savings Reports, facilitates scheduling of communication between pharmacists and prescribers, including syncing appointments with calendars within the DSR portal, outside calendars, or schedules within EHRs.

The system and method for dynamic interactive DSR services allows administrators to view all activities and reporting in a system Dashboard view, including DSRs sent and acted upon that includes resultant savings to the Payer. Reporting capabilities include DSRs requested, DSRs delivered, actions taken on each DSR, aggregate actions taken categorized by Pharmacists or group of Pharmacists, by Payor and by prescriber or prescriber groups. Dashboard functionality includes administrator-set automatic notifications, such as a delay in delivering requested DSRs.

Disclosed herein are various embodiments of a non-transitory, computer-readable, storage medium storing a set of instructions capable of being executed by a processor to perform operations for prescription alternatives identification and management, and that when executed by the processor, causes the processor to intercept an electronic request by Pharmacist to generate one or more DSRs and then deliver them via a portal. The non-transitory, computer-readable, storage medium storing a set of instructions that when executed by the processor, can cause the processor to identify a plurality of alternative medications for each medication selected by the Pharmacist. The non-transitory, computer-readable, storage medium storing a set of instructions capable of being executed by a processor, performs operations for prescription alternatives identification and management, and can further rank or prioritize the identified alternatives based upon interactive rules specified by, or determined by, the actions of Payers and/or providers. It can further cause the processor to submit an inquiry transaction to a PBM of the Patient for a plurality of medication alternatives, receive a response of the inquiry transactions from the PBM, determine the most cost effective of the alternatives, and present these alternatives to the Pharmacists via the portal.

For example, described herein are methods of generating a drug savings report (and systems configured to perform these methods) that include: receiving, in a configurable alternatives engine (CAE), a plurality of requests to identify lower-cost alternative prescribed medications for a plurality of patients; generating, in a Data Matching and Scoring module, a linked list of patient identifiers specific to each patient of the plurality of patients, wherein, for each patient of the plurality of patients, the patient identifiers correspond to a different third-party database of a plurality of different third-party databases comprising: an electronic health record (EHR) database, a database of pharmacy claims, and a pharmacy benefit manager (PBM); formulating, in a Data Query and Transfer module, a plurality of queries specific for each different third-party database and specific to each patient, using the linked list of patient identifiers; aggregating a subset of the plurality of queries specific for each different third-party database and specific to each patient into a batch query for each of the different third-party databases; submitting each batch query to a corresponding third-party database on a submission scheduled, wherein the submission scheduled is based on a scheduling protocol of the Data Query and Transfer module, so that each batch query is delayed by the Data Query and Transfer module until a time indicated by the submission scheduling; receiving responses from the batch query for each third-party database and correlating responses specific to each patient using the linked list of patient identifiers; generating, in the CAE, a patient-specific datastore for each patient using the received responses: a set of the patient's preexisting prescribed medications, a plurality of therapeutically equivalent alternative medications corresponding to the patient's preexisting prescribed medications, and a total cost associated with the subset of the therapeutically equivalent alternative medications, wherein the total cost includes both an estimated patient financial responsibility and an estimated Payer financial responsibility; and outputting a drug savings report (DSR) listing alternatives for the patient's preexisting prescribed medications, wherein the DSR shows the patient's savings for each of the therapeutically equivalent alternative medications in which at least one of: the patient cost of the therapeutically equivalent alternative medication is lower than the patient cost of the corresponding preexisting prescribed medication or the total cost of the therapeutically equivalent alternative medication is lower than the total cost of the corresponding preexisting prescribed medication.

Any of these methods may include identifying, from the Payer Preferred Alternative Medications Database and the Prescriber Preferred Database of the CAE, a plurality of therapeutically equivalent alternative medications corresponding to the patient's preexisting prescribed medications in the set based on a clinical equivalency, wherein a Payer is responsible for financing health care services for the patient.

The methods described herein may include formulating a second plurality of queries specific for each different third-party database and specific to each patient, using the linked list of patient identifiers and the received responses and modifying the patient-specific datastore for each patient base on responses to the second plurality of queries.

In any of these methods aggregating the subset of the plurality of queries may be done by the Data Query and Transfer module. In some examples aggregating the subset of the plurality of queries specific for each different third-party database comprises aggregating queries for the PBM to get a patient cost and a total cost associated with the subset of the therapeutically equivalent alternative medications, wherein the total cost includes both an estimated patient financial responsibility and an estimated Payer financial responsibility.

In any of these methods a scheduling protocol of the Data Query and Transfer module may include a list of periodically updated times of day in which one or more of the third party databases are less expensive to use. The scheduling protocol of the Data Query and Transfer module may include a list of periodically updated times of day in which one or more of the third party databases receive a lower density of queries.

For example, a system for generating a drug savings report may include: one or more processors; a memory coupled to the one or more processors, the memory storing computer-program instructions, that, when executed by the one or more processors, perform a computer-implemented method comprising: receiving, in a configurable alternatives engine (CAE), a plurality of requests to identify lower-cost alternative prescribed medications for a plurality of patients; generating, in a Data matching and Scoring module, a linked list of patient identifiers specific to each patient of the plurality of patients, wherein, for each patient of the plurality of patients, the patient identifiers correspond to a different third-party database of a plurality of different third-party databases comprising: an electronic health record (EHR) database, a database of pharmacy claims, and a pharmacy benefit manager (PBM); formulating, in a Data Query and Transfer module, a plurality of queries specific for each different third-party database and specific to each patient, using the linked list of patient identifiers; aggregating a subset of the plurality of queries specific for each different third-party database and specific to each patient into a batch query for each of the different third-party databases; submitting each batch query to a corresponding third-party database on a submission scheduled, wherein the submission scheduled is based on a scheduling protocol of the Data Query and Transfer module, so that each batch query is delayed by the Data Query and Transfer module until a time indicated by the submission scheduling; receiving responses from the batch query for each third-party database and correlating responses specific to each patient using the linked list of patient identifiers; generating, in the CAE, a patient-specific datastore for each patient using the received responses: a set of the patient's preexisting prescribed medications, a plurality of therapeutically equivalent alternative medications corresponding to the patient's preexisting prescribed medications, and a total cost associated with the subset of the therapeutically equivalent alternative medications, wherein the total cost includes both an estimated patient financial responsibility and an estimated Payer financial responsibility; and outputting a drug savings report (DSR) listing alternatives for the patient's preexisting prescribed medications, wherein the DSR shows the patient's savings for each of the therapeutically equivalent alternative medications in which at least one of: the patient cost of the therapeutically equivalent alternative medication is lower than the patient cost of the corresponding preexisting prescribed medication or the total cost of the therapeutically equivalent alternative medication is lower than the total cost of the corresponding preexisting prescribed medication.

The computer-program instructions may further comprises identifying, from the Payer Preferred Alternative Medications Database and the Prescriber Preferred Database of the CAE, a plurality of therapeutically equivalent alternative medications corresponding to the patient's preexisting prescribed medications in the set based on a clinical equivalency, wherein a Payer is responsible for financing health care services for the patient.

In some examples the computer-program instructions further comprises formulating a second plurality of queries specific for each different third-party database and specific to each patient, using the linked list of patient identifiers and the received responses and modifying the patient-specific datastore for each patient base on responses to the second plurality of queries.

The computer-program instructions may further be configured to aggregate the subset of the plurality of queries is done by the Data Query and Transfer module. In some examples, the computer-program instructions may be further configured to aggregate the subset of the plurality of queries specific for each different third-party database comprises aggregating queries for the PBM to get a patient cost and a total cost associated with the subset of the therapeutically equivalent alternative medications, wherein the total cost includes both an estimated patient financial responsibility and an estimated Payer financial responsibility. The scheduling protocol of the Data Query and Transfer module may comprise a list of periodically updated times of day in which one or more of the third party databases are less expensive to use. For example, the scheduling protocol of the Data Query and Transfer module may include a list of periodically updated times of day in which one or more of the third party databases receive a lower density of queries.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a flowchart illustrating one example of a method of generating a drug savings report (DSR) including aggregating multiple patient requests, as described herein.

### DETAILED DESCRIPTION

Described herein are methods (e.g., computer-implemented methods) and systems (including software) for generating (including automatically generating) drug savings reports (DSRs) for multiple patients.

As used herein, “Acute Medication” means a Medication that is taken on a one-time basis and is not generally taken again or over an extended period of time. The methods and apparatuses described herein may be configured to identify and acute medications and to characterize them distinctly from recurring medications, which may further streamline the processes described herein.

As used herein “API” means an Application Programing Interface. Any of the methods and systems described herein may be used with or may include and API.

As used herein, “Alternate Fulfillment” means an alternate pharmacy or site of service where Medications may be dispensed or provided other than the Member's preferred pharmacy or site of service that is selected by the provider in the Electronic Health Record. This may include mail service and specialty pharmacy providers.

As used herein “alternate Medication” means a therapeutically equivalent alternate to the Existing Medication (prescription or over-the-counter, or medical equipment/device used with or used to administer a patient, beneficiary or health plan member.

As used herein an “existing Medication” means Maintenance or Periodic Medication or medical equipment/device previously prescribed or documented by the provider or staff as identified in claims files or by the electronic health record.

As used herein “claims” may refer, depending on the context, to electronic or paper pharmacy claims that are transmitted to Claims Adjudicator by Participating Pharmacies, Members, or Payer as a result of Covered Prescription Drug Services being supplied to Members by Participating Pharmacies, with the result being that the claims are either paid, denied, rejected or reversed.

As used herein, “CMS-4180” Means the Modernizing Part D and Medicare Advantage To Lower Drug Prices and Reduce Out-of-Pocket Expenses Rule by the Centers for Medicare & Medicaid Services on May 23, 2019.

As used herein, “completed” Transactions are the respective DSR completed transactions where the amount of the Member Costs, the alternatives to Existing Medication, and the Total Savings for Alternate Medications are delivered to a member.

As used herein, a “configurable Alternatives Engine” or “CAE” may refer to a system or sub-system (including software, firmware and/or hardware) for electronically determining and providing therapeutically equivalent Alternate Medications for an Existing Medication, and that allows ongoing interactive and rules-based configuration and customization of the Alternatives presented based upon Provider or Payer preferences. As described herein a CAE may control access to the various databases (third-party databases) referencing patient information, pricing information, drug alternative information, insurer information, physician information, pharmacist information, etc.

As used herein, “Drug Savings Report” or “Drugs and Savings Report” or “DSR” refers to an output (e.g., of a CAE) that identifies medications or medical equipment/devices currently prescribed for a member along with therapeutically equivalent lower cost Alternate Medications and assemble these medications and related costs and savings into a single report or display for viewing by prescribers, pharmacists, members or other authorized individuals or entities. DSRs are delivered to prescribers integrated into EHRs (“DSR-E”), to pharmacists and other authorized users via online Gemini portal (“DSR-D”) and to members (“DSR-M”) as specified by the Payer. The DSR service also provides a medication list based upon pharmacy claims for review by healthcare providers to promote medication reconciliation and patient safety.

As used herein, a Drug Savings Reports Direct (“DSR-D”) is an interactive service wherein a user, often a pharmacist, requests, receives, customizes, forwards and works upon one or more DSRs. A Drug Savings Reports in EHRs (“DSR-E”) is a service that automatically queries and delivers DSRs into EHRs often triggered by a patient appointment in a medical office or clinic. A Drug Savings Report for Members (“DSR-M”) is a single DSR created for a member that may be delivered directly to the member or through an authorized intermediary.

As used herein a “DSR Transaction” is a completed transaction with all claims adjudications and costs and savings returned to a member for a single DSR request, whether or not there are multiple drugs in the DSR. A single DSR Transaction request is the electronic transmission of a request for an adjudication of a claim by the Claims Adjudicator against the Member's specific defined benefit and formulary and the corresponding claim adjudication response, error codes, error messages, Prior Authorization flags and other Payer- or remote server-specified fields to the remote server using the most current NCPDP Telecommunications Standard Version (transaction standard) or other format as agreed upon. The DSR Transaction may be triggered by a request.

As used herein “EOB” means an explanation of benefits related to prescription drug coverage.

As used herein, “Electronic Health Record” or “EHR” is the electronic version of a patient's medical history maintained by the provider over time that includes key administrative clinical data relevant to that person's care under a particular provider, and that uses application programming interfaces (API) to provide other care-related activities including clinical decision support and electronic prescribing. It includes systems with only electronic prescribing functionality (i.e., “standalone” ePrescribing systems).

As used herein, “Maintenance Medication” means a Medication that is taken routinely (e.g., daily or twice a day) over an extended period of time.

As used herein, “Medications” includes a drug, vaccine, or medical device used for medical treatment. Medications are categorized as Acute Medications, Maintenance Medications and Periodic Medications.

As used herein, a “Member Cost” means the out-of-pocket financial responsibility that the Member incurs for Medications. The Member Cost may include costs incurred for certain Covered Prescription Drug Services provided under the Plan (copayment, coinsurance, or other costs as defined by the Plan); full cost or the discounted pharmacy network cost of prescription Medications for those who must meet a deductible, are in a High Deductible Health Plan, or for prescription Medications not covered or excluded by the Plan; or costs incurred to fulfill the balance of a deductible and costs under the Plan.

As use herein, a “Medication Previously Taken” (MPT) means a prescription medication previously taken by a patient but currently not included in the patient's active medication list or regimen.

As used herein a “National Council for Prescription Drug Programs” or “NCPDP” means the ANSI-accredited, standards development organization. A NCPDP may be one type of remote database accessed by the methods and systems described herein.

As used herein a “PBM” means a pharmacy benefits manager. A PBM may be one type of remote database accessed by the methods and systems described herein.

As used herein a “Periodic Medication” means a Medication that is taken on an as-needed or episodic basis over an extended period of time.

As used herein a “Plan” may refer to a health plan, employer or other sponsor that includes prescription drug benefits for Members.

As used herein, a “Prescriber” means the person who is licensed to prescribe prescription Medications, approved by the U.S. Food and Drug Administration (FDA), for patients.

As used herein, a “prescription” broadly means drugs, medications, compounds, vaccines, medical devices, therapies, or anything a healthcare provider can order for a Patient.

As used herein, a “Shared Patient Savings” (SPS) means a payment or reward provided to a patient by a Plan to increase the patient's financial motivation to collaborate with their providers to select the most cost-effective medications.

Health costs continue to grow in the U.S. and are well documented as a formidable national challenge, with prescription drug costs among the leading sources of cost escalation. For Patients taking prescription drugs, these costs can be materially addressed by interactively identifying lower cost therapeutic equivalent alternative drug options in the context of a strategy that is sometimes referred to as “cost transparency” for patients and their care-givers (“Patient”). Patients, however, are unable to change their prescriptions without a licensed prescriber. Prescribers, typically doctors, have limited time and limited information available to select lower cost medications, and may have limited experience with some medications that may be lower cost for patients. Pharmacists, population health clinicians or other providers (“Pharmacists”) have the scope of knowledge, expertise and responsibility to assist in identifying lower cost drugs for patients however, they lack robust interactive tools to request, receive, customize, prioritize and act upon lower cost drug alternatives.

Currently, there is no alternative drug or fulfillment cost information that is available on a routine or automated basis related to the cost-effectiveness of existing medications. This is due, in part, to the complexity of the health care system, and particularly the medication component of the health care system. There are numerous systems controlled or mediated by insurers, hospitals, pharmacies, health care providers, pharmaceutical suppliers, and government agencies, that all contribute toward the billing and reimbursement of mediations. Each of these systems may include information that are typically accessed in an ad-hoc manner. Each of these systems may include different access restrictions and costs. In particular, accessing databases such as PBMs, insurer databases, patient electronic health records, pharmacy systems, and the like, may be expensive, and separate rules and guidelines may be provided for accessing each of these. For example, these systems may charge for some types of queries (and/or some information) but not all information or types of queries. In addition, some of these systems may charge fees based on the time of day, or may impose penalties based on the number total number of queries sent. In addition, each of these databases may require a different format for accessing and receiving information. Described herein are system and methods that provide a technical solution to the problem posed by these diverse and distributed systems, allowing the information to be collected and processed in order to provide transparency in drug pricing and alternatives.

Prescribers are typically notified to renew existing prescriptions for their Patients by phone call, fax or electronic systems when the existing prescription has expired—but cost information or equivalent alternative drugs of lower cost are not typically provided to the Prescriber, even if the cost of the medication—to the payer, employer or health plan (“Payer”) and the Patient—have gone up dramatically. In addition, prescribers often delegate renewals to clinical office staff who do not have the ability to change medications to lower cost alternatives even if presented during the renewal process. Prescriber's primary job is to focus on patient care, not drug costs.

Pharmacists who work with Prescribers are often tasked with identifying and recommending lower cost alternatives for Patients but have no related interactive tools to proactively identify these alternatives and identify savings on a Patient-specific basis. These Pharmacists may receive flat files including spread sheets that list current Patient medications. Separately they may have static lists of formularies relevant to groups of Patients. As for selecting alternatives to existing medications Pharmacists lack an interactive system that identifies all potential alternatives including mapping daily doses and day's supply. Patient-specific drug costs and cost savings opportunities are not currently part of Pharmacists workflow for existing prescriptions. For these reasons, while Pharmacists attempt to reduce the costs for existing medications, their ability is limited by the lack of a robust interactive tools; and as a result, drug costs continue to escalate.

For most drug categories there are numerous drug alternatives that a Prescriber might consider in treating a Patient though a Prescriber may not have experience with all alternatives. Also, many drugs are used for more than one diagnosis making the selection of alternatives more challenging since Pharmacists and payers may not know the diagnosis being treated for each patient. Pharmacists are familiar with all drugs and alternatives but lack Patient-specific costs that include the Patient's pharmacy benefits coverage, formularies, deductibles and copays. Pharmacists are therefore unable to give Prescribers a list of alternatives to consider, nor the cost of alternative drugs, so both clinical and economic efficiency are lacking. Even if Pharmacists are presented alternatives, they are unable to customize these alternatives to include drugs which they prefer or exclude alternatives in general, on behalf of a prescriber and/or on a patient-specific basis. Also, they lack the ability to change potential alternative drugs based upon the history or diagnosis being treated for each patient.

Drug costs can vary by many factors including: drug chosen, form of drug (tablet vs capsule, cream vs ointment, extended release, etc.), Patient's insurance, Patient's deductible-including high deductible plans and Affordable Care Act (ACA) health plans, Pharmacy and Patient financial assistance programs. Drug costs are dynamic and change all the time—even between new scripts and renewal scripts. In addition, insured Patients may find that their drug costs are lowered by using alternative means for fulfillment. For example, $4 Generics (Wal-Mart, Target, etc.), Pharmacy Discount Card or mail order, etc. The determination of the pricing of a medication can be complicated.

The optimal alternative drug, at the optimal cost, for a given Patient may vary substantially based upon Payer formularies, Payer preferences, Pharmacists preferences, Prescriber preferences, patient clinical status and also medications previously taken (“MPT”) by the patient.

In current Pharmacist workflow the Pharmacist, working for a medical group or Payer, is tasked with lowering the drug costs for a population of patients under the care of Prescribers. The provider group may have entered into an Accountable Care Organization (“ACO”) contract with a payer or other type of contract that puts the provider group at risk for the overall pharmacy spend or otherwise rewards provider groups for lowering drug cost. Yet, aside from simple payer pharmacy claims, the Pharmacists lack the interactive tools to proactively identify and customize lower cost alternatives to existing medications that are patient and payer specific including diagnosis and patient-specific drug costs. Absent these tools Pharmacists struggle to do their job and drug costs continue to rise.

While Prescribers have shown an interest in knowing drug costs, alternative drugs and their costs, and related clinical messages about the prescriptions to be created or renewed (e.g., prior Authorization required, alternative drugs to consider, etc.), Prescribers do not want to spend time considering all possible therapeutic alternatives and then price-shopping for each Patient. Prescribers look to Pharmacists to assist in identifying lower cost alternatives that are optimized for each patient.

Therefore, there is a pressing need for an interactive system and method for the identification and selecting therapeutic drug alternatives for existing medications including taking in to account costs, as well as Payer formularies, Payer preferences, Pharmacists preferences, Prescriber preferences, including preferred means and times of engagement, patient clinical status including diagnosis if available, and also medications previously taken (“MPT”) by the patient, and delivering the alternatives information to Pharmacists in workflow, enabling them to then engage Prescribers with recommended alternatives. There is a related need to provide simple interactive Prescriber-Pharmacists communications related to lower cost alternatives in a manner that takes into account prescriber preferred means and times of engagement eliminates to the extent possible the Prescriber's administrative tasks of contacting pharmacies and patients to implement the switch to a lower cost alternative. There is an additional need to provide information back to Pharmacists confirming that existing drugs were in-fact switched to lower cost alternatives by identifying the alternatives in the Payer's claims file. There is also a need to send updated information regarding lower cost therapeutic drug alternatives to Pharmacists based upon changes in the patient's medications, lower cost therapeutic drug alternatives available including new drugs, drugs going off patent, changes in formularies, changes in rebates, changes in pharmacy coverage and members, changes in providers who are members of a medical group, or a payer, member or provider-defined time interval.

The following descriptions of the various examples of the disclosure are not intended to limit the invention to these examples, but rather to enable any person skilled in the art to make and use this invention. While the present disclosure has been disclosed in example embodiments, those of ordinary skill in the art will recognize and appreciate that many additions, deletions, and modifications to the disclosed embodiments and their variations may be implemented without departing from the scope of the disclosure.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

The systems, devices, and methods of the preferred embodiments and variations thereof can be embodied and/or implemented at least in part as a machine configured to receive a computer-readable medium storing computer-readable instruction. The instructions are preferably executed by computer-executable components preferably integrated with the system including the computing device configured with software. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

In some examples, a system for dynamic interactive Drug Savings Report (“DSR”) services (e.g., generation) may include all of some of the following logical components: Current Medication Data, Clinical Content Data, Payer Formulary Data, Payer Pharmacy Claims Data, Payer Member Eligibility Data, Provider group Data and NPIs, Data Query and Transfer, Data Matching and Scoring, Provider or Provider Group Medication Preferences Data, Configurable Alternatives Engine software, rules and databases, and Interactive Portal. Each of these components may be configured as a module including one or more engine and/or data structure.

Thus, any of these systems can be implemented one or more modules, including one or more engines, or a part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

With respect to the systems for dynamic interactive Drug Savings Report (“DSR”) services (e.g., generation) described herein, these systems may include a Current medication modulate for processing and/or determining one or a plurality (including a large number of patient's) current medication data. For example, current medication data represents medications currently taken by the patient on an ongoing basis as listed in the EHR or as reflected in payer claims. Thus, a current medication data may compare claims-based and EHR-based medication lists for reconciliation and/or patient safety purposes.

Any of these systems may include a Configurable Alternatives Engine (“CAE” or CAE module), which may provide prescription alternative identification and management over time for the dynamic generation of Drug Savings Reports data repository used to store clinical content. The CAE may be used to identify clinically appropriate, dose adjusted, alternative medications and corresponding medication Quantities and Days' Supply.

A Clinical Content Data module may support the following: identification of whether a medication is recognized as Maintenance or Periodic medication, identification of whether a medication is recognized as Multi-Source or Single Source, identification of whether a medication is recognized as a Brand or Generic sourced drug, identification of whether a medication is available in multiple forms which may impact costs (tablet vs capsule, cream vs ointment, extended release, etc.), identification of clinically aligned medications via therapeutic category, identification of clinically aligned medications via diagnosis code (when appropriate), identification of conversion factors that enables the software to identify dose adjusted Alternative Medications, Quantities and Days' Supply, identification of cost information that enables the software to calculate an Estimated Cost for the Chosen Medication and each Alternative Medication, and identification of a Representative NDC, either Brand or Generic, including dosage and form (i.e., tablet vs capsule, cream vs ointment) for each Alternative Medication category.

For example a clinical content data may include one or more agents (including, but not limited to a machine learning agent) that may automatically identify all or some of these components. In particular, the methods and systems described herein may identify some or all of the clinical content data.

The methods and systems described herein may also or alternatively include a Payer Formulary Data module. The Payer Formulary Data module may identify and manage (over time) the Prescription alternative(s) used in dynamic generation of Drug Savings Reports data repository used to store Payer formulary content. Payer Formulary Data may identify if a medication is covered, or not covered, and the Payer's relative preference for one medication over another medication within a therapeutic category. Payer Formulary Data (and the Payer Formulary Data module) may be used by the Configurable Alternatives Engine component to align identified Alternative Medications with the Patient's pharmacy benefit formulary by ranking the medications.

Payer Formulary Data may be an alternative to ranking Alternative Medications via Estimated Cost and used with a Payer Formulary or Default Formulary. Payer Formulary Data is delivered to the server from participating PBMs” or directly from contracted Payers. The receipt and processing of Payer Formulary Data may be an automated process.

Payer rebate files may also be used to better estimate total savings from alternative drugs.

A Payer Pharmacy Claims Data module may be a repository that stores pharmacy transaction data, both billing and reversal transactions, delivered to the Payer's claims adjudication processing engine by a contracted pharmacy. Payer Pharmacy Claims Data may be used to identify medications filled by the patient, billed to the Payer and subsequently dispensed. If the final transaction is a billing transaction the medication was billed, dispensed, and acquired by the member. If the final transaction is a reversal transaction the medication was returned to stock and not acquired by the member. Pharmacy Claims Data may be used to create and maintain Member Medication Lists and also determine whether an alternative medication was prescribed. Member Medication Lists may identify the patient current medications.

Payer Pharmacy Claims Data may be used by the systems and methods described herein to identify the Patient's medication list and can be also used to compare medication lists to the prescriber's Electronic Health Record (EHR) medication list.

Payer Pharmacy Claims Data is delivered to remove server (e.g., the server performing the methods described herein) via the Payer or the Payer's PBM or pharmacy claims processor agent. The receipt and processing of Payer Pharmacy Claims Data is an automated process.

A Provider Group Data and NPIs module may include a database of medical providers who are members of a specific medical group using the DSR services. Providers can join a group or leave a group making this database dynamic. The introduction of a new provider may trigger the need for dynamic generation of Drug Savings Reports.

Any of these methods and apparatuses (e.g., systems) may include a Data Query and Transfer module that formulates the data queries for the various third-party databases. The Data Query and Transfer module may formats, group and/or schedule the requests for data (queries) used by the system, including any or all of the modules described herein. For example, a Data Query and Transfer module may include logic configured to acquire all or some of: Current Medication Data, Clinical Content Data, Payer Formulary Data, Payer Pharmacy Claims Data, Payer Member Eligibility Data, and Provider group Data and non-pharmaceutical interventions (NPIs). In some examples one or more third party database may be queried and data transferred based upon DSR requests. Thus, the query and transfer may involve multiple platforms, high volume data transfer, multiple data formats, and requires high utilization of system resources. The methods and apparatuses described herein may be particularly configured to optimize this process.

In practice, the third party databases (e.g., data sources) are typically used by a multitude of other (e.g., unrelated) entitles, including, in particular, use for real time active users for other purposes. In order to optimize system resources and comply with data holder system usage requirements, the methods and apparatuses described herein may combine (e.g., batch) queries, that may then be queued and processed utilizing scheduling logic that optimizes system and data holder resources. Batch schedule processing frees system resources to complete individual real time requests needed on a priority basis. In some examples the queries be combined in a manner that reduces overlapping requests for information.

Queries may be scheduled by the Data Query and Transfer module so that a particular number of queries (e.g., less than a maximum threshold, or in some examples, more than a minimum threshold) are submitted together. The scheduling may be based on feedback or information from the third-party database, which may include and/or be based on usage or traffic data. For example, the Data Query and Transfer module may include, receive or generate a schedule of low-traffic and/or low-cost periods specific to each third-party database (e.g., PBM, etc.) within which batched queries may be transmitted to each different third-party database. The schedule may be specific to low-volume/low traffic periods. In some cases the Data Query and Transfer module may first request from some or all of the third-party databases if the database is in an operating mode that is low-cost (e.g., during off hours).

As mentioned, the Data Query and Transfer module may batch or collect requests for information specific to groups of patients. The group of patients may be unrelated or related. For example, the Data Query and Transfer module may group or batch together patients that share one or more similar features (e.g., medication type, medical group, insurer, etc.) or it may batch unrelated groups of patients.

Any of the systems described herein may also include a Data Matching and Scoring module, which may be part of the Data Query and Transfer module or may be a separate module. The Data Matching and Scoring module may generate and maintain a linked list of patients and query information. For example, the Data Matching and Scoring module may generate for each patient identifying information specific to each patient that is derived from the patient information provided by the original request (e.g., a DSR request). For each third-party database a separate patient identifier (e.g., patient identifying code, etc.) may be required. The Data Matching and Scoring may generate a data structure of patient identifier specific to each third party database and this data structure, linking each patient to patient identifier and specific third party database, may be used both for preparing queries as well as for receiving data from the third party databases in response to each query. An enormous volume of data is generated through these DSR queries/requests. DSR data from multiple platforms (multiple third party databases) and sources can be unified into a consolidated patient data structure using a dynamic matching logic that is able to identify, link, and organize these multiple data sources. Further, the sheer quantity of data being communicated may otherwise hamper utilization of this data. The system, including the Data Matching and Scoring module, can utilize scoring logic to prioritize the data based on the highest opportunity for savings utilizing a series of system developed metrics. The resulting data presentation allows for a better utilization of resources toward the greatest opportunity for success.

Also described herein is a Provider or Provider Group Medication Preferences Module. The Provider or Provider Group (“Providers”) Medication Preferences module may include Prescription alternative identification and management over time including dynamic generation of Drug Savings Reports data repository used by the Configurable Alternatives Engine component to identify a Providers' medication preferences, if any and applicable. Preferences may be identified and configured manually or automatically through claims data or cost transparency transactions, including alternatives selected by Providers and those not selected. Prescription alternative identification and management over time, including dynamic generation of Drug Savings Reports, may support three Providers Medication Preferences scenarios: (i.) Passes an indicator to Transaction Processing Services when the Chosen Medication is a Providers Preferred Medication, (ii.) Passes an indicator to the Transaction Processing Services when an Alternative Medication is a Provider Preferred Medication, or (iii.) Replaces an identified Alternative Medication with a Providers Preferred Medication.

In any of these examples, the Payer may approve the preferred medications. The Payer or the Provider Group may identify the preferred medications. The Configurable Alternatives Engine (“CAE”) may be configured to include the Alternative Therapy Messaging module, which may receive an Alternative Therapy Request with Chosen Medication, Quantity, and Days' Supply data and pharmacy benefit information (when Patient has pharmacy benefit insurance) and optionally an assigned diagnosis either with the original request or with a subsequent request after an alternative diagnosis has been identified. The Alternative Therapy Messaging module may also intersect the Chosen Medication, Quantity, & Days' Supply information and business rules to the Clinical Content data. The Alternative Therapy Messaging module may also intersect the identified Alternative Medications and the Patient's pharmacy benefit information (i.e., BIN Number, Processor Control Number, Group ID, and Cardholder ID) with the corresponding Payer Formulary Data to rank the Alternative Medications, and may identify up to three up Alternative Medications based upon clinical equivalency, payer costs, formularies and rules and provider preferences if any. In some examples, the Alternative Therapy Messaging module may pass a Cost Transparency Response with up to three selected Alternative Medications to the Cost Transparency Processing component.

In any of these methods and systems, the Configurable Alternatives Engine may intercept the data and apply the rules that identify the Alternative Medications which may include noting which alternatives are medications previously taken (“MPT”), including the date of last fill.

In some examples the Configurable Alternatives Engine interact with databases that contain drugs available in multiple forms that impact costs such as tablet vs capsule, cream vs ointment, extended release, etc. to optimize the alternative selected.

The Cost Transparency Response data may be used to format and deliver PBM Request transactions to the Patient's Pharmacy Benefit Manager or a Drug Savings Card processor.

Also described herein are Interactive Portals for preparing or presenting the drug saving report (DSR), and/or reports. For example, an Interactive Portal may be the Alternative Therapy component that allows Pharmacists, Prescribers, Patients or other authorized provider to perform one or more of: register for DSR Portal services, including communication and payment preferences, request DSRs or be notified of DSRs that are ready to be viewed or discussed with pharmacists or other Portal users; view, triage, note as pending, or download DSRs; modify alternatives in DSRs and trigger the DSRs to be regenerate with new alternative drugs; customize alternatives to include Pharmacist or provider preferences, including alternatives that should always be presented in response to an existing medication or never presented either in general or for a specific patient; see if a requested DSR has been requested and viewed in the past and, if so, when and by whom with what recommendations resulting; see if a specific drug in a DSR has been changed from a past DSR for the same Patient; have DSRs in pending files be automatically flagged for the Pharmacists based upon Pharmacist-selected timing or rules; have DSRs autogenerated for the Pharmacist based upon new Prescribers in their group, new patients under the care of the Prescribers including changes during open enrollment, new medications for patients, changes in Payer formularies including rebated drugs; communicate DSRs to Prescribers via fax, email, into the prescriber's EHR in an interactive manner, or other electronic means as specified by the Prescriber; receive DSRs once reviewed by the prescriber with switches to lower cost alternatives authorized by the prescriber allowing the pharmacists to change the prescription and the pharmacy and notify the patient of the change; identify Prescriber preferences including; previously switched medications changes, never switched medication changes or preferred dates, times and means of communication of recommended alternative switches; and/or communicate specific lower cost alternatives recommended to Prescribers or Patients for confirmation via the portal that the recommended switch was confirmed via Payer claims.

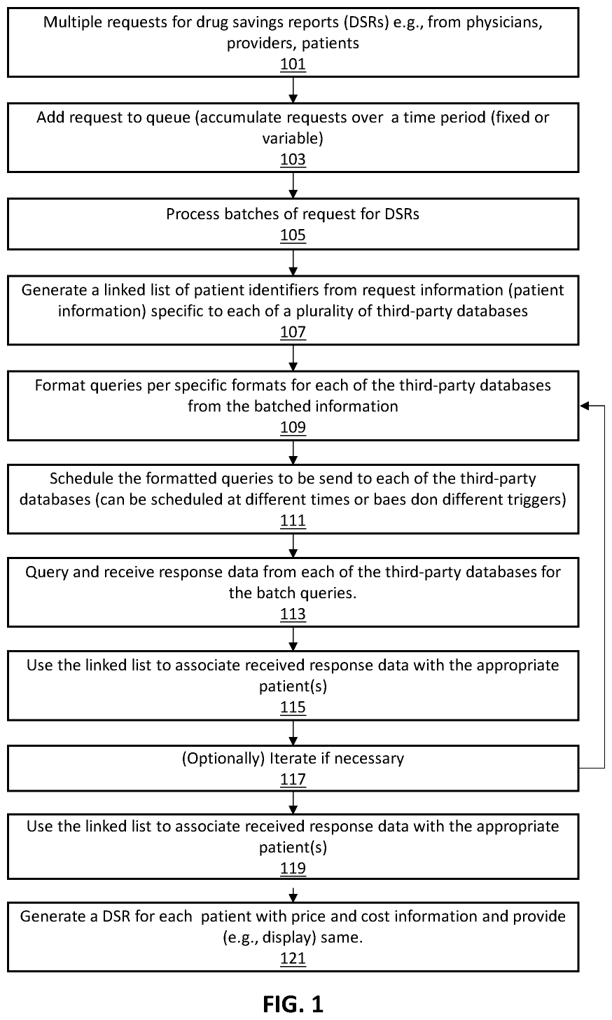

For example, FIG. 1 illustrates one method of generating drug savings reports for multiple patients as described herein. In FIG. 1, one or more requests are received by a system 101 for DSRs, e.g., from physicians, patients, providers, etc. The requests may be received by a remote processor that is configured to perform these steps and to generate and provide the DSRs. For example, request may be aggregated by the remote server 103. In some examples, multiple requests (e.g., approximately 10, 50, 100, 150, 200, 500, 1000, 1500, 2000, 2500, 3000, 3500, 4000, 5000, 6000, 7000, 8000, 9000, 10,000, 15,000 or more, etc. requests may be aggregated. These DSR requests may be batched 105 and may be processed together. In some examples, the requests may be pre-processed to aggregate similar requests (e.g., same physician, same insurer, etc.) together.

The system or method may then generate a linked list of patients and patient identifiers 107. Each linked list may include patient identifiers that are specific to the different types of third-party databases; the patient identifiers may be generated by the system or method using information supplied with the request or accessible from the request (e.g., patient health record information).

In general, to assemble a drug saving report (or DSR), data from multiple sources may be selected base on specific patient details. The system may include information about the third-party system, including query format, patient identifier format, and the like.

Thus, the system may format the queries for each third-party system (third-party database) from the batched information, including patients 109. To assemble the DSR information, the system or method may schedule the formatted queries to be sent 111 based on the usage and/or cost of each of the various third-party databases (including but not limited to the PBM). The system may allow the queries to be scheduled as data pulls to be made during off hours and/or when usage is otherwise low 113. This may not only reduce the costs, but may also significantly reduce traffic. Thus, any of these queries may be delayed (not in real time) based on this usage detail and/or cost of the third party database.

Once the data of the batched queries has been received from the multiple sources (the multiple third-party databases), the data may be associated with each of the specific patients in the batch 115 fusing the linked list.

The data may be analyzed to provide a DSR; in some cases, the method or system my include repeating nay of these steps 117. Further, the system may unpack the batched information received using the linked list 119. Finally a GSR may be generated from the received data by applying the methods outlined in this application 121. The linking data may provide the ability to take multiple data sources and aggregated them, by providing a key for transmitting and receiving. System receives from the physician—the electronic health record for the patient—they make a request—the patient is in the system, can provide it as part of the query.

In general, any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of generating a drug savings report, the method comprising:
receiving, in a configurable alternatives engine (CAE), a plurality of requests to identify lower-cost alternative prescribed medications for a plurality of patients;
generating, in a Data Matching and Scoring module, a linked list of patient identifiers specific to each patient of the plurality of patients, wherein, for each patient of the plurality of patients, the patient identifiers correspond to a different third-party database of a plurality of different third-party databases comprising: an electronic health record (EHR) database, a database of pharmacy claims, and a pharmacy benefit manager (PBM);
formulating, in a Data Query and Transfer module, a plurality of queries specific for each different third-party database and specific to each patient, using the linked list of patient identifiers;
aggregating a subset of the plurality of queries specific for each different third-party database and specific to each patient into a batch query for each of the different third-party databases;
submitting each batch query to a corresponding third-party database on a submission scheduled, wherein the submission scheduled is based on a scheduling protocol of the Data Query and Transfer module, so that each batch query is delayed by the Data Query and Transfer module until a time indicated by the submission scheduling;
receiving responses from the batch query for each third-party database and correlating responses specific to each patient using the linked list of patient identifiers;
generating, in the CAE, a patient-specific datastore for each patient using the received responses: a set of the patient's preexisting prescribed medications, a plurality of therapeutically equivalent alternative medications corresponding to the patient's preexisting prescribed medications, and a total cost associated with the subset of the therapeutically equivalent alternative medications, wherein the total cost includes both an estimated patient financial responsibility and an estimated Payer financial responsibility; and
outputting a drug savings report (DSR) listing alternatives for the patient's preexisting prescribed medications, wherein the DSR shows the patient's savings for each of the therapeutically equivalent alternative medications in which at least one of: the patient cost of the therapeutically equivalent alternative medication is lower than the patient cost of the corresponding preexisting prescribed medication or the total cost of the therapeutically equivalent alternative medication is lower than the total cost of the corresponding preexisting prescribed medication.

2. The method of claim 1, further comprising identifying, from the Payer Preferred Alternative Medications Database and the Prescriber Preferred Database of the CAE, a plurality of therapeutically equivalent alternative medications corresponding to the patient's preexisting prescribed medications in the set based on a clinical equivalency, wherein a Payer is responsible for financing health care services for the patient.

3. The method of claim 1, further comprising formulating a second plurality of queries specific for each different third-party database and specific to each patient, using the linked list of patient identifiers and the received responses and modifying the patient-specific datastore for each patient base on responses to the second plurality of queries.

4. The method of claim 1, wherein aggregating the subset of the plurality of queries is done by the Data Query and Transfer module.

5. The method of claim 1, wherein aggregating the subset of the plurality of queries specific for each different third-party database comprises aggregating queries for the PBM to get a patient cost and a total cost associated with the subset of the therapeutically equivalent alternative medications, wherein the total cost includes both an estimated patient financial responsibility and an estimated Payer financial responsibility.

6. The method of claim 1, wherein the scheduling protocol of the Data Query and Transfer module comprises a list of periodically updated times of day in which one or more of the third party databases are less expensive to use.

7. The method of claim 1, wherein the scheduling protocol of the Data Query and Transfer module comprises a list of periodically updated times of day in which one or more of the third party databases receive a lower density of queries.

8. A system for generating a drug savings report, the system comprising:
one or more processors;
a memory coupled to the one or more processors, the memory storing computer-program instructions, that, when executed by the one or more processors, perform a computer-implemented method comprising:
receiving, in a configurable alternatives engine (CAE), a plurality of requests to identify lower-cost alternative prescribed medications for a plurality of patients;
generating, in a Data matching and Scoring module, a linked list of patient identifiers specific to each patient of the plurality of patients, wherein, for each patient of the plurality of patients, the patient identifiers correspond to a different third-party database of a plurality of different third-party databases comprising: an electronic health record (EHR) database, a database of pharmacy claims, and a pharmacy benefit manager (PBM);
formulating, in a Data Query and Transfer module, a plurality of queries specific for each different third-party database and specific to each patient, using the linked list of patient identifiers;
aggregating a subset of the plurality of queries specific for each different third-party database and specific to each patient into a batch query for each of the different third-party databases;
submitting each batch query to a corresponding third-party database on a submission scheduled, wherein the submission scheduled is based on a scheduling protocol of the Data Query and Transfer module, so that each batch query is delayed by the Data Query and Transfer module until a time indicated by the submission scheduling;
receiving responses from the batch query for each third-party database and correlating responses specific to each patient using the linked list of patient identifiers;
generating, in the CAE, a patient-specific datastore for each patient using the received responses: a set of the patient's preexisting prescribed medications, a plurality of therapeutically equivalent alternative medications corresponding to the patient's preexisting prescribed medications, and a total cost associated with the subset of the therapeutically equivalent alternative medications, wherein the total cost includes both an estimated patient financial responsibility and an estimated Payer financial responsibility; and
outputting a drug savings report (DSR) listing alternatives for the patient's preexisting prescribed medications, wherein the DSR shows the patient's savings for each of the therapeutically equivalent alternative medications in which at least one of: the patient cost of the therapeutically equivalent alternative medication is lower than the patient cost of the corresponding preexisting prescribed medication or the total cost of the therapeutically equivalent alternative medication is lower than the total cost of the corresponding preexisting prescribed medication.

9. The system of claim 8, wherein the computer-program instructions further comprises identifying, from the Payer Preferred Alternative Medications Database and the Prescriber Preferred Database of the CAE, a plurality of therapeutically equivalent alternative medications corresponding to the patient's preexisting prescribed medications in the set based on a clinical equivalency, wherein a Payer is responsible for financing health care services for the patient.

10. The system of claim 8, wherein the computer-program instructions further comprise formulating a second plurality of queries specific for each different third-party database and specific to each patient, using the linked list of patient identifiers and the received responses and modifying the patient-specific datastore for each patient base on responses to the second plurality of queries.

11. The system of claim 8, wherein the computer-program instructions are further configured to aggregate the subset of the plurality of queries is done by the Data Query and Transfer module.

12. The system of claim 8, wherein the computer-program instructions are further configured to aggregate the subset of the plurality of queries specific for each different third-party database comprises aggregating queries for the PBM to get a patient cost and a total cost associated with the subset of the therapeutically equivalent alternative medications, wherein the total cost includes both an estimated patient financial responsibility and an estimated Payer financial responsibility.

13. The system of claim 8, wherein the scheduling protocol of the Data Query and Transfer module comprises a list of periodically updated times of day in which one or more of the third party databases are less expensive to use.

14. The system of claim 8, wherein the scheduling protocol of the Data Query and Transfer module comprises a list of periodically updated times of day in which one or more of the third party databases receive a lower density of queries.
